(19) United States
(12) Patent Application Publication
(10) Pub. No.: US 2010/0266518 A1
(43) Pub. Date: Oct. 21, 2010

(54) Process for the preparation of odourless polyether alcohols using DMC catalysts and their use in cosmetic and/or dermatological preparations

(75) Inventors: Oliver Springer, Wesel (DE); Oliver Thum, Ratingen (DE); Frank Schubert, Neukirchen-Vluyn (DE)

Correspondence Address: FROMMER LAWRENCE & HAUG, 745 FIFTH AVENUE- 10TH FL., NEW YORK, NY, 10151, US

(73) Assignee: EVONIK GOLDSCHMIDT GMBH, Essen (DE)

(21) Appl. No.: 12/759,787

(22) Filed: Apr. 14, 2010

(30) Foreign Application Priority Data
Apr. 15, 2009 (DE) 102009002371.2

(51) Int. Cl. A61K 8/34; A61Q 17/04; C07C 43/13; A61K 47/08; A61Q 15/00

(52) U.S. Cl. 424/59; 424/65; 514/772; 568/679

(57) ABSTRACT

Cosmetic and/or dermatological preparations prepared using polyether alcohols, characterized in that, for the preparation of the polyether alcohol,
a) a DMC catalyst is used and
b) the polyether alcohols obtained in this way are not further treated.

This application claims benefit under 35 U.S.C. 119(a) of German patent application DE 10 2009 002 371.2, filed on Apr. 15, 2009.

Any foregoing applications including German patent application DE 10 2009 002 371.2, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Polyether alcohols, often also simply referred to in short as polyethers, have been known for a long time and are produced industrially in large amounts. They are used, inter alia, as surfactants, emulsifiers or foam suppressants. The desired properties can be established here in a targeted manner through the type of alcohol and the type and amount of the polyether fraction. Pure propylene oxide based polyether alcohols are also used as oil phase in cosmetic preparations, inter alia AP/Deo formulations (=antiperspirant/deodorant formulations).

Most processes for the preparation of alkoxylation products (polyethers) use basic catalysts such as, for example, the alkali metal hydroxides and the alkali metal methylates. The use of KOH or NaOH is particularly widespread and has been known for many years. Typically, a mostly low molecular weight hydroxy-functional starter (starting alcohol) such as butanol, allyl alcohol, propylene glycol or glycerol is reacted in the presence of the alkaline catalyst with an alkylene oxide such as ethylene oxide, propylene oxide, butylene oxide or a mixture of different alkylene oxides to give a polyoxyalkylene polyether. The strongly alkaline reaction conditions during this so-called living polymerization promote various secondary reactions. Particular disadvantages are the complex product work-up caused by neutralization of the alkaline polymer (see e.g. U.S. Pat. No. 3,715,402, U.S. Pat. No. 4,430,490, U.S. Pat. No. 4,507,475 and U.S. Pat. No. 4,137,398) and the base-catalysed rearrangement, which proceeds as a secondary reaction, of epoxides, for example propylene oxide, to give allyl alcohols or propenyl alcohols. These propenyl polyethers have proven to be an undesired source of olfactory product contaminants in cosmetic preparations—as the result of the hydrolytic lability of the vinyl ether bond present therein and release of propionaldehyde. A process for the preparation of low-odour polyether polyols is described, inter alia, in EP 1 062 263 (U.S. Patent Appl. Pub. 2002-183560).

Disadvantages of the base-catalysed alkoxylation without doubt include the need to free the resulting reaction products from the active base with the help of a neutralization step. Distillative removal of the water which is formed during the neutralization, as well as removal of the salt formed by filtration are then mandatory.

Besides the base-catalysed reaction, acidic catalyses for the alkoxylation are also known. Thus, DE 10 2004 007561 (U.S. Patent Appl. Pub. 2007-185353) describes the use of HBF₄ and of Lewis acids such as, for example, BF₃, AlCl₃ and SnCl₄ in alkoxylation technology.

A disadvantage of the acid-catalysed polyether synthesis has proven to be the defective regioselectivity during the ring opening of asymmetrical oxiranes such as, for example, propylene oxide, which leads to polyoxyalkylene chains with both secondary and primary OH termini being obtained in a manner which cannot be controlled in a definitive manner. As in the case of the base-catalysed alkoxylation reaction, here too, a work-up sequence of neutralization, distillation and filtration is imperative. If ethylene oxide is introduced as monomer into the acid-catalysed polyether synthesis, then the formation of dioxane as undesired by-product should be expected.

However, the catalysts used for the preparation of polyether alcohols are also often multimetal cyanide compounds or double metal cyanide catalysts, commonly also referred to as DMC catalysts. The use of DMC catalysts minimizes the content of unsaturated by-products; moreover, compared with the customary basic catalysts, the reaction proceeds with a significantly higher space-time yield. The preparation and use of double metal cyanide complexes as alkoxylation catalysts has been known since the 1960s and is depicted, for example, in U.S. Pat. No. 3,427,256, U.S. Pat. No. 3,427,334, U.S. Pat. No. 3,427,335, U.S. Pat. No. 3,278,457, U.S. Pat. No. 3,278,458, U.S. Pat. No. 3,278,459. Among the ever more effective types of DMC catalysts that were further developed in subsequent years and are described e.g. in U.S. Pat. No. 5,470,813 and U.S. Pat. No. 5,482,908, are specifically zinc-cobalt hexacyano complexes. Thanks to their extraordinary high activity, only small catalyst concentrations are required for the preparation of polyetherols, meaning that it is possible to dispense with the work-up step required for conventional alkaline catalysts—consisting of the neutralization, precipitation and removal of the catalyst by filtration—at the end of the alkoxylation process. The alkoxylation products prepared using DMC catalysts are characterized by a much narrower molar mass distribution compared to alkali-catalysed products. It is attributed to the high selectivity of the DMC-catalysed alkoxylation that, for example, propylene-oxide-based polyethers contain only very small fractions of unsaturated by-products.

The alkoxylation reaction carried out, in direct comparison with alkali and acid catalysis, over DMC catalysts is, among the described technical characteristics, so advantageous that it has led to the development of continuous processes for the preparation of high-volume simple polyetherols that consist mostly only of PO units.

For example, WO 98/03571 (U.S. Pat. No. 5,689,012) describes a process for the continuous preparation of polyether alcohols using DMC catalysts, in which, in a continuous stirred vessel, firstly a mixture of a starter and a DMC catalyst is initially introduced, the catalyst is activated, and further starter, alkylene oxides and DMC catalysts are continuously added to this activated mixture and, after achieving the desired fill level in the reactor, polyether alcohol is continuously drawn off.

EP 1 756 198 (U.S. Patent Appl. Pub. 2008-033214) describes a process for the preparation of low-odour polyetherpolyols by means of DMC catalysis, the polyethers also being treated by means of stripping gas.

WO 01/62826 (U.S. Pat. No. 6,673,972), WO 01/62824 (U.S. Pat. No. 7,022,884) and WO 01/62825 (U.S. Pat. No. 6,664,428) describe specific reactors for the continuous process for the preparation of polyether alcohols using DMC catalysts.

In the case of the industrial processes described here, the patent literature is directed in particular to the monodispersity of the polyetherol obtained by DMC processes. Thus, narrow molar mass distributions are often desired, as in the case of the polyols used for PU foaming systems (DE 100 08630, U.S. Pat. No. 5,689,012).

However, a low molar mass distribution is not synonymous with high quality in all fields of application. In sensitive applications, an excessively low polydispersity may even be a disadvantage, which restricts the usability of DMC-based polyether/polyether alcohols. For example, the specification EP-A-1066334 (U.S. Pat. No. 6,066,683) points in this connection to the fact that the polyether alcohols obtained by an alkaline alkoxylation process cannot be substituted in a simple manner by the polyetherols prepared by means of DMC catalysis. The usefulness of the polyether alcohols obtained via DMC catalysis and characterized by their narrow molecular weight distribution is limited particularly where they are used as one component in a multicomponent system. This multicomponent system may be, inter alia, a cosmetic preparation.

### DESCRIPTION OF THE INVENTION

It is an object of the invention to provide raw materials for cosmetic and/or dermatological preparations which are able to at least replace known raw materials and in so doing no longer have the stability and olfactory problems of the raw materials in the prior art.

This applies particularly to cosmetic and/or dermatological preparations in the antiperspirant/deodorant category, in particular in so-called “stick formulations”, which often still exhibit the problem of inadequate stability, insofar as odour changes arise during storage.

Surprisingly, it has been found that polyetherols which are prepared by means of DMC catalysis at very low catalyst concentrations can be used without further treatment steps in cosmetic and/or dermatological preparations, in particular AP/Deo preparations, without exhibiting undesired odour developments, even after a prolonged storage time.

With the preparations according to the invention, it is possible to replace known polyetherols which have been prepared by acid- or base-catalysed processes and which have by-products which have to be removed in a complex manner by means of neutralization, filtration and/or distillation steps.

The polyetherols prepared by the DMC process (double metal cyanide process for the alkoxylation) no longer have these disadvantages, can be used directly without purification and neutralization and can be exchanged as a direct replacement for the known compounds.

With the DMC process, it is possible, depending on the epoxide used and type of epoxide ring opening, to prepare compositions comprising polyether alcohols of the formula (I):

R¹—[O-(A)ₙ-(B)ₘ—H]ₓ (I)
R¹ being a linear or branched alkyl radical having 3 to 22 carbon atoms,
A independently of B being an identical or different ethyleneoxy, propyleneoxy, butyleneoxy, styreneoxy, cyclohexyloxy unit or a unit originating from a glycidyl compound as the result of epoxide ring opening,
B independently of A being an identical or different ethyleneoxy, propyleneoxy, butyleneoxy, styreneoxy, cyclohexyloxy unit or a unit originating from a glycidyl compound as a result of epoxide ring opening,
m being equal to 0 to 20,
n being equal to 1 to 40,
x being an integer from 1 to 6,
   and the monomer units A and B can be strung together in their order arbitrarily either blockwise or randomly.

In particular, with the process, it is possible to synthesize compositions comprising polyethers of the formula (I) which are characterized in that they can be prepared in a targeted and reproducible manner in terms of structural composition and molar mass distribution, do not have to be worked-up and permit odourless cosmetic and/or dermatological preparations. The cosmetic and/or dermatological preparations can contain 0.1 to 80% by weight of the polyethers of the formula (I). The amount varies depending on the further additives used and the desired hardness or viscosity of the target formulation, for example a deodorant stick.

The prior art refers to alkoxylation processes which use catalysis with double metal cyanide catalysts. By way of reference, mention may be made here, for example, of EP-A-1017738 (U.S. Pat. No. 6,077,978), U.S. Pat. No. 5,777,177, EP-A-0981407 (U.S. Pat. No. 5,844,070), WO 2006/002807 (US Pub. 2007-0225394) and EP-A-1474464 (U.S. Pat. No. 7,312,363).

In the reaction mixture, the catalyst concentration is preferably >0 to 1000 wppm (mass ppm), preferably >0 to 500 wppm, particularly preferably 0.1 to 100 wppm and very particularly preferably 1 to 50 wppm. Here, this concentration is based on the total mass of the polyetherpolyols formed; the reaction temperature is about 60 to 250° C., preferably from 90 to 160° C. and particularly preferably about 100 to 130° C. The pressure at which the alkoxylation takes place is preferably 0.02 bar to 100 bar, preferably 0.05 to 20 bar absolute.

Alcohols R¹OH which can be used in the process according to the invention are in particular monofunctional alcohols having 4 to 22 carbon atoms, preferably 4 to 18 carbon atoms, such as butanol, myristyl alcohol and stearyl alcohol.

Within the context of the invention, epoxide monomers which can be used are, besides ethylene oxide, propylene oxide, butylene oxide, styrene oxide, 1,2-dodecene oxide and cyclohexene oxide, all known further mono- and polyfunctional epoxide compounds including the glycidyl ethers and esters, individually or in a mixture and either randomly or else in a block-like order.

To start the reaction, it may be advantageous if firstly a reaction mixture which contains the DMC catalyst, if desired slurried in a suspending agent, is initially introduced into the reactor and at least one alkylene oxide is metered into this. The molar ratio of alkylene oxide to reactive groups, in particular OH groups, in the starting mixture is preferably 0.1 to 5:1, preferably 0.2 to 2:1. It may be advantageous if, prior to the addition of the alkylene oxide, any substances which inhibit the reaction that are present are removed from the reaction mixture, e.g. by distillation. Suspending agents which can be used are either a polyether or inert solvents or advantageously also the starting compound onto which the alkylene oxide is to be added, or a mixture of the two.

The start of the reaction can be detected, for example, by monitoring the pressure. A sudden drop in pressure in the reactor in the case of gaseous alkylene oxides indicates that the alkylene oxide is incorporated, the reaction has thus started and the end of the starting phase has been reached.

After the starting phase, that is to say after initialization of the reaction, depending on the desired molar mass, either starting compound and alkylene oxide are metered in simultaneously, or only alkylene oxide. Alternatively, it is also possible to add any desired mixture of different alkylene oxides. The reaction can be carried out in an inert solvent, for example for the purpose of lowering the viscosity. The molar ratio of the metered alkylene oxides, based on the starting compound used, in particular based on the number of OH groups in the starting compound used, is here preferably expressed as the sum of the indices n+m equal to 1 to 60. Preferably, propylene oxide is always only used in a mixture with a further alkylene oxide.

Within the context of the present invention, starting compounds are understood as meaning substances which form the start of the polyether molecule to be prepared which is obtained through the addition reaction of alkylene oxide. The starting compound used in the process according to the invention is preferably selected from the group of alcohols, polyetherols, phenols or carboxylic acids. As starting compound, preference is given to using a mono- or polyhydric polyether alcohol or alcohol R¹—OH.

The OH-functional starting compounds used are preferably compounds with molar masses from 18 to 1000 g/mol, in particular 100 to 2000 g/mol and 1 to 6, preferably 1 to 4, hydroxyl groups. By way of example, mention may be made of butanol, hexanol, octanol, 2-ethylhexanol, nonanol, isononanol, decanol, undecanol, dodecanol, 2-butyloctanol, tridecanol, tetradecanol, hexadecyl alcohol, 2-hexyldecanol, stearyl alcohol, isostearyl alcohol or behenyl alcohol. Moreover, mixtures of the aforementioned alcohols are suitable.

Low molecular weight polyetherols with 1-6 hydroxyl groups and molar masses of from 100 to 2000 g/mol which have for their part previously been prepared by DMC-catalysed alkoxylation are advantageously used as starter compounds.

Besides compounds with aliphatic and cycloaliphatic OH groups, any desired compounds with 1-20 phenolic OH functions are suitable. These include, for example, phenol, alkyl- and arylphenols, bisphenol A and novolacs.

Reactors which can be used in principle for the reaction claimed according to the invention are all suitable reactor types which allow the reaction and its possible exothermicity to be controlled.

The reaction management can take place in a manner known in process technology continuously, semi-continuously or else batchwise and can be adjusted flexibly to the production technology equipment present.

Besides conventional stirred-tank reactors, it is also possible to use jet loop reactors with gas phase and external heat exchangers, as described, for example, in EP-A-0 419 419, or internal heat exchanger pipes, as described in WO 01/62826. Moreover, gas-phase-free loop reactors can be used.

During the metered addition of the starting materials, good distribution of the substances involved in the chemical reaction, i.e. of the alkylene oxides and/or glycidyl compounds, starters, DMC catalysts and, if desired, suspending agents is necessary.

After the alkylene oxide addition and possible afterreaction to complete the alkylene oxide conversion, the product can be worked up. The work-up required here involves in principle only the removal of unreacted alkylene oxide and possibly further, readily volatile constituents, usually by vacuum distillation, steam stripping or gas stripping or other methods of deodorization. The removal of readily volatile secondary components can take place either batchwise or continuously. In the case of the process based on DMC catalysis, it is possible, in contrast to the conventional base-catalysed alkoxylation, in the standard case to dispense with a filtration.

If necessary, it is possible to remove the DMC catalyst from the finished polyether alcohol. However, for most fields of use, it can remain in the polyether alcohol. In principle, it is possible, although not preferred, to separate off the DMC catalyst and to use it again, as described, for example, in WO 01/38421. However, this procedure is in most cases too complex for the large-scale industrial production of polyether alcohols.

It is customary to stabilise the formed polyether alcohol against thermooxidative degradation. This usually takes place by adding stabilizers, in most cases sterically hindered phenols such as, for example, BHT—butylhydroxytoluene, BHA—butylhydroxyanisole, TBHQ—tert-butylhydroquinone or penta-erythritol tetrakis(3-(3,5-di-tert-butyl-4-hydroxyphenyl)-propionate).

The addition reaction of the alkylene oxide compounds or more generally expressed, epoxide compounds, preferably takes place at a temperature of from 60 to 250° C., preferably from 90 to 160° C. and particularly preferably at a temperature from 100 to 130° C. The pressure at which the alkoxylation takes place is preferably 0.02 bar to 100 bar, preferably 0.05 to 20 bar absolute. By carrying out the alkoxylation at subatmospheric pressure, the reaction can be carried out very safely. If desired, the alkoxylation can be carried out in the presence of an inert gas (e.g. nitrogen) and also at superatmospheric pressure.

The process steps can be carried out at identical or different temperatures. The mixture initially introduced into the reactor at the reaction start and comprising starting substance, and DMC catalyst can be pretreated in accordance with the teaching of WO 98/52689 (U.S. Pat. No. 5,844,070) by stripping prior to the start of the metered addition of the alkylene oxides. Here, an inert gas is admixed with the reaction mixture via the reactor feed and, with the help of a vacuum apparatus attached to the reactor system, more readily volatile components are removed from the reaction mixture by applying a subatmospheric pressure. In this simple manner it is possible to remove substances from the reaction mixture which can inhibit the catalyst, such as e.g. lower alcohols or water. The addition of inert gas and the simultaneous removal of the more readily volatile components can be advantageous particularly at the start of the reaction since inhibiting compounds can also pass into the reaction mixture through the addition of the reactants or as a result of secondary reactions.

DMC catalysts which can be used are all known DMC catalysts, preferably those which have zinc and cobalt, preferably those which have zinc hexacyanocobaltate(III). Preference is given to using the DMC catalysts described in U.S. Pat. No. 5,158,922, US 20030119663, WO 01/80994 (U.S. Pat. No. 6,835,687) or in the aforementioned specifications. The catalysts may be amorphous or crystalline.

In the reaction mixture, the catalyst concentration is preferably >0 to 1000 wppm (mass ppm), preferably >0 to 500 wppm, particularly preferably 0.1 to 100 wppm and very particularly preferably 1 to 50 wppm. Here, this concentration is based on the total mass of the polyetherpolyols.

Preferably, the catalyst is only metered into the reactor once. The amount of catalyst should be adjusted to ensure adequate catalytic activity for the process. The catalyst can be metered as solid or in the form of a catalyst suspension. If a suspension is used, then in particular the starting polyether is suitable as suspension agent. Preferably, however, a suspending operation is dispensed with.

The cosmetic preparations can comprise, for example, at least one additional component selected from the group of

emollients,
emulsifiers and surfactants,
thickeners/viscosity regulators/stabilizers,
UV photoprotective filters,
antioxidants and vitamins,
hydrotropes (or polyols),
solids and fillers,
film formers,
pearlescent additives,
deodorant and antiperspirant active ingredients,
esterase inhibitors,
insect repellents,
self-tanning agents,
preservatives,
conditioners,
perfumes,
dyes,
biogenic active ingredients,
care additives,
superfatting agents,
solvents.

Emollients which can be used are all cosmetic oils, in particular mono- or diesters of linear and/or branched mono- and/or dicarboxylic acids having 2 to 44 carbon atoms with linear and/or branched saturated or unsaturated alcohols having to 22 carbon atoms. It is likewise possible to use the esterification products of aliphatic, difunctional alcohols having 2 to 36 carbon atoms with monofunctional aliphatic carboxylic acids having 1 to 22 carbon atoms. Also suitable are long-chain aryl acid esters, such as, for example, esters of benzoic acid, e.g. benzoic acid esters of linear or branched, saturated or unsaturated alcohols having 1 to 22 carbon atoms, or else isostearyl benzoate or octyldodecyl benzoate. Further monoesters suitable as emollients and oil components are, for example, the methyl esters and isopropyl esters of fatty acids having 12 to 22 carbon atoms, such as, for example, methyl laurate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and esters which are obtainable from technical-grade aliphatic alcohol cuts and technical-grade, aliphatic carboxylic acid mixtures, e.g. esters of unsaturated fatty alcohols, having 12 to 22 carbon atoms and saturated and unsaturated fatty acids having 12 to 22 carbon atoms, as are accessible from animal and vegetable fats. Also suitable, however, are naturally occurring monoester and/or wax ester mixtures, as are present, for example in jojoba oil or in sperm oil. Suitable dicarboxylic acid esters are, for example, di-n-butyl adipate, di-n-butyl sebacate, di(2-ethylhexyl) adipate, di(2-hexyldecyl) succinate, diisotridecyl azelate. Suitable diol esters are, for example, ethylene glycol dioleate, ethylene glycol diisotridecanoate, propylene glycol di(2-ethyl-hexanoate), butanediol diisostearate, butanediol dicaprylate/caprate and neopentyl glycol dicaprylate. Further fatty acid esters which can be used as emollients are, for example, C₁₂-₁₅ alkyl benzoate, dicaprylyl carbonate, diethylhexyl carbonate. Emollients and oil components which can likewise be used are longer-chain triglycerides, i.e. triple esters of glycerol with three acid molecules, of which at least one is relatively long-chain. By way of example, mention may be made here of fatty acid triglycerides; examples of such which may be used are natural, vegetable oils, e.g. olive oil, sunflower oil, soybean oil, peanut oil, rapeseed oil, almond oil, sesame oil, avocado oil, castor oil, cocoa butter, palm oil, but also the liquid fractions of coconut oil or of palm kernel oil, and also animal oils, such as, for example, shark liver oil, cod liver oil, whale oil, beef tallow and butter fat, waxes such as beeswax, carnauba palm wax, spermaceti, lanolin and claw oil, the liquid fractions of beef tallow and also synthetic triglycerides of caprylic/capric acid mixtures, triglycerides of technical-grade oleic acid, triglycerides with isostearic acid, or from palmitic acid/oleic acid mixtures as emollients and oil components. Furthermore, hydrocarbons, in particular also liquid paraffins and isoparaffins, can be used. Examples of hydrocarbons which can be used are paraffin oil, isohexadecane, polydecene, vaseline, Paraffinum perliquidum, squalane, ceresine. Furthermore, it is also possible to use linear or branched fatty alcohols such as oleyl alcohol or octyldodecanol, and also fatty alcohol ethers such as dicaprylyl ether. Suitable silicone oils and silicone waxes are, for example, polydimethylsiloxanes, cyclomethylsiloxanes, and also aryl- or alkyl- or alkoxy-substituted polymethylsiloxanes or cyclomethylsiloxanes. Suitable further oil bodies are, for example, Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched C₈-C₁₈-alcohols, in particular 2-ethylhexanol or isononanol, esters of branched C₆-C₁₃-carboxylic acids with branched alcohols, in particular 2-ethylhexanol or isononanol, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, vegetable oils, branched primary alcohols, substituted cyclohexanes, linear C₆-C₂₂-fatty alcohol carbonates, Guerbet carbonates, esters of benzoic acid with linear and/or branched C₆-C₂₂-alcohols, dialkyl ethers, ring-opening products of epoxidized fatty acid esters with polyols, silicone oils and/or aliphatic or naphthenic hydrocarbons.

The oil bodies/emollients (polyetherpolyols according to the invention plus further oil bodies) are usually present in a total amount of 0.1-90% by weight, in particular 0.1-80% by weight, in particular 0.5 to 70% by weight, preferably 1 to 60% by weight, in particular 1 to 40% by weight and preferably 5 to 25% by weight. The further oil bodies are usually present in an amount of from 0.1 to 40% by weight, based on the total weight of the preparation.

Emulsifiers or surfactants which may be used are nonionic, anionic, cationic or amphoteric surfactants.

Nonionogenic emulsifiers or surfactants which can be used are compounds from at least one of the following groups:

addition products of from 2 to 100 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C_{12/18}-fatty acid mono- and diesters of addition products of from 1 to 100 mol of ethylene oxide onto glycerol,
glycerol mono- and diesters and sorbitan mono- and diesters of saturated and unsaturated fatty acids having 6 to 22 carbon atoms and ethylene oxide addition products thereof,
alkyl mono- and oligoglycosides having 8 to 22 carbon atoms in the alkyl radical and ethylene oxide addition products thereof, addition products of from 2 to 200 mol of ethylene oxide onto castor oil and/or hydrogenated castor oil,
partial esters based on linear, branched, unsaturated or saturated C₆-C₂₂-fatty acids, ricinoleic acid, and 12-hydroxy-stearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose),
mono-, di- and trialkylphosphates, and mono-, di- and/or tri-PEG alkyl phosphates and salts thereof,
polysiloxane-polyether copolymers (dimethicone copolyols), such as, for example PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 4/12 or 20/20 or 18/18 or 17/18 or 15/15,
polysiloxane-polyalkyl-polyether copolymers and corresponding derivatives, such as, for example, lauryl or cetyl dimethicone copolyols, in particular cetyl PEG/PPG-10/1 dimethicone (ABIL® EM 90 (Evonik Goldschmidt GmbH)),
mixed esters of pentaerythritol, fatty acids, citric acid and fatty alcohol as in DE 11 65 574 (GB 962919) and/or mixed esters of fatty acids having 6 to 22 carbon atoms, methylglucose and polyols, such as, for example, glycerol or polyglycerol,
citric acid esters, such as, for example, glyceryl stearate citrate, glyceryl oleate citrate and dilauryl citrate.

Anionic emulsifiers or surfactants can contain water-solubilizing anionic groups, such as, for example, a carboxylate, sulphate, sulphonate or phosphate group and a lipophilic radical. Skin-compatible anionic surfactants are known to the person skilled in the art in large numbers and are commercially available. Here, these may be alkyl sulphates or alkyl phosphates in the form of their alkali metal, ammonium or alkanolammonium salts, alkyl ether sulphates, alkyl ether carboxylates, acyl sarcosinates, and sulphosuccinates and acyl glutamates in the form of their alkali metal or ammonium salts.

Cationic emulsifiers and surfactants can also be added. Those which can be used are, in particular, quaternary ammonium compounds, in particular those provided with at least one linear and/or branched, saturated or unsaturated alkyl chain having 8 to 22 carbon atoms, such as, for example, alkyltrimethylammonium halides, such as, for example, cetyltrimethylammonium chloride or bromide or behenyltrimethylammonium chloride, but also dialkyldimethylammonium halides, such as, for example, distearyldimethylammonium chloride.

Furthermore, monoalkylamidoquats such as, for example, palmitamidopropyltrimethylammonium chloride or corresponding dialkylamidoquats, can be used.

Furthermore, readily biodegradable quaternary ester compounds can be used; these may be quaternized fatty acid esters based on mono-, di- or triethanolamine. Furthermore, alkylguanidinium salts can be added as cationic emulsifiers.

Typical examples of mild, i.e. particularly skin-compatible, surfactants are fatty alcohol polyglycol ether sulphates, monoglyceride sulphates, mono- and/or dialkyl sulphosuccinates, fatty acid isethionates, fatty acid sarcosinates, fatty acid taurides, fatty acid glutamates, ether carboxylic acids, alkyl oligoglucosides, fatty acid glucamides, alkylamidobetaines and/or protein fatty acid condensates, the latter for example based on wheat proteins.

Furthermore, it is possible to use amphoteric surfactants, such as, for example, betaines, amphoacetates or amphopropionates, thus, for example, substances such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethyl-imidazolines having in each case 8 to 18 carbon atoms in the alkyl or acyl group, and also cocoacylaminoethyl hydroxyethyl-carboxymethyl glycinate.

Of the ampholytic surfactants, it is possible to use those surface-active compounds which, apart from a C_{8/18}-alkyl or -acyl group in the molecule, contain at least one free amino group and at least one —COOH— or —SO₃H— group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkyl-aminoacetic acids having in each case about 8 to 18 carbon atoms in the alkyl group. Further examples of ampholytic surfactants are N-cocoalkylaminopropionate, cocoacylaminoethyl-aminopropionate and C_{12/18}-acylsarcosine.

The preparations according to the invention comprise the emulsifier(s) and/or surfactants usually in an amount of from 0 to 40% by weight, preferably 0.1 to 20% by weight, preferably 0.1 to 15% by weight and in particular 0.1 to 10% by weight, based on the total weight of the preparation.

Suitable thickeners are, for example, polysaccharides, in particular xanthan gum, guar guar, agar agar, alginates and tyloses, carboxymethylcellulose and hydroxyethylcellulose, also relatively high molecular weight polyethylene glycol mono- and diesters of fatty acids, polyacrylates (e.g. Carbopols TM or Synthalens TM), polyacrylamides, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates with a narrowed homologue distribution or alkyl oligoglucosides, and also electrolytes such as sodium chloride and ammonium chloride.

Suitable thickeners for thickening oil phases are all thickeners known to the person skilled in the art. In particular, mention is to be made here of waxes, such as hydrogenated castor wax, beeswax or microwax. Furthermore, inorganic thickeners can also be used, such as silica, alumina or sheet silicates (e.g. hectorite, laponite, saponite). In this connection, these inorganic oil phase thickeners may be hydrophobically modified. For the thickening/stabilization of water-in-oil emulsions, in particular aerosils, sheet silicates and/or metal salts of fatty acids, such as, for example, magnesium stearate, aluminium stearate and/or zinc stearate, or magnesium ricinoleate, aluminium ricinoleate and/or zinc ricinoleate, can be used here.

Viscosity regulators for aqueous surfactant systems which may be present are, for example NaCl, low molecular weight nonionic surfactants, such as cocoamide DEA/MEA and laureth-3, or polymeric, high molecular weight, associative, highly ethoxylated fat derivatives, such as PEG-200 hydrogenated glyceryl palmate.

UV photoprotective filters which can be used are, for example, organic substances which are able to absorb ultraviolet rays and which give off the absorbed energy again in the form of longer-wave radiation, e.g. heat. UVB filters may be oil-soluble or water-soluble. Examples of oil-soluble UVB photoprotective filters are:

3-benzylidenecamphor and derivatives thereof, e.g. 3-(4-methyl-benzylidene)camphor, 4-aminobenzoic acid derivatives, such as, for example, 2-ethylhexyl 4-(dimethylamino)benzoate,
2-ethylhexyl 4-(dimethylamino)benzoate and amyl 4-(dimethylamino)benzoate, esters of cinnamic acid, such as 2-ethylhexyl 4-methoxycinnamate, isopentyl 4-methoxycinnamate, 2-ethylhexyl 2-cyano-3-phenylcinnamate (octocrylene), esters of salicylic acid, such as, for example, 2-ethylhexyl salicylate, 4-isopropylbenzyl salicylate, homomethyl salicylate,
   derivatives of benzophenone, such as, for example, 2-hydroxy-4-methoxybenzophenone, 2-hydroxy-4-methoxy-4′-methylbenzophenone, 2,2′-dihydroxy-4-methoxybenzophenone, esters of benzalmalonic acid, such as, for example, di-2-ethylhexyl 4-methoxy-benzmalonate,
   triazine derivatives, such as, for example, 2,4,6-trianilino(p-carbo-2′-ethyl-1′-hexyloxy)-1,3,5-triazine, octyltriazone and those described in EP 1180359 and DE 2004/027475,
   propane-1,3-diones, such as, for example, 1-(4-tert-butylphenyl)-3-(4′-methoxyphenyl)propane-1,3-dione.

Suitable water-soluble UVB photoprotective filters are:

2-phenylbenzimidazole-5-sulphonic acid and the alkali metal, alkaline earth metal, ammonium, alkylammonium, alkanolammonium and glucammonium salts thereof,
sulphonic acid derivatives of benzophenone, such as, for example, 2-hydroxy-4-methoxybenzophenone-5-sulphonic acid and its salts,
sulphonic acid derivatives of 3-benzylidenecamphor, such as, for example, 4-(2-oxo-3-bornylidenemethyl)benzenesulphonic acid and 2-methyl-5-(2-oxo-3-bornylidene)sulphonic acid and salts thereof.

Suitable typical UVA photoprotective filters are in particular derivatives of benzoylmethane, such as, for example, 1-(4′-tert-butylphenyl)-3-(4′-methoxyphenyl)propane-1,3-dione or 1-phenyl-3-(4′-isopropylphenyl)propane-1,3-dione. The UV-A and UV-B filters can of course also be used in mixtures.

Besides the specified soluble substances, insoluble pigments, namely finely disperse metal oxides or salts are also suitable for this purpose, such as, for example, titanium dioxide, zinc oxide, iron oxide, aluminium oxide, cerium oxide, zirconium oxide, silicates (talc), barium sulphate and zinc stearate. The particles here should have an average diameter of less than 100 nm, e.g. between 5 and 50 nm and in particular between 15 and 30 nm. They can have a spherical shape, although it is also possible to use those particles which have an ellipsoidal shape or a shape which deviates in some other way from the spherical form. A relatively new class of photoprotective filters are micronized organic pigments, such as, for example, 2,2′-methylenebis{6-(2H-benzotriazol-2-yl)-4-(1,1,3,3-tetramethyl-butyl)phenol} with a particle size of <200 nm, which is obtainable, for example, as 50% strength aqueous dispersion.

Further suitable UV photoprotective filters can be found in the overview by P. Finkel in SÖFW-Journal 122, 543 (1996).

The preparations according to the invention can comprise the UV photoprotective filters in amounts of from 0 to 30% by weight, preferably 0 to 20% by weight, based on the total weight of the preparation.

Besides the two aforementioned groups of primary UV photoprotective filters, it is also possible to use secondary photoprotective agents of the antioxidant type which interrupt the photochemical reaction chain which is triggered when UV radiation penetrates into the skin.

Antioxidants and vitamins which can be used are, for example, superoxide-dismutase, tocopherol (vitamin E), tocopherol sorbate, tocopherol acetate, other esters of tocopherol, dibutylhydroxytoluene and ascorbic acid (vitamin C) and its salts, and also derivatives thereof (e.g. magnesium ascorbyl phosphate, sodium ascorbyl phosphate, ascorbyl sorbate), ascorbyl esters of fatty acids, butylated hydroxybenzoic acid and its salts, peroxides, such as, for example, hydrogen peroxide, perborates, thioglycolates, persulphate salts, 6-hydroxy-2,5,7,8-tetramethylchroman-2-carboxylic acid (TROLOX®), gallic acid and its alkyl esters, uric acid and its salts and alkyl esters, sorbic acid and its salts, lipoic acid, ferulic acid, amines (e.g. N,N-diethylhydroxylamine, aminoguanidines), sulfhydryl compounds (e.g. glutathione), dihydroxy-fumaric acid and its salts, glycine pidolate, arginine pidolate, nordihydroguaiaretic acid, bioflavonoids, curcumin, lysine, L-methionine, proline, superoxide dismutase, silymarin, tea extract, grapefruit peel/pip extract, melanin, rosemary extract, thioctanoic acid, resveratrol, oxyresveratrol, etc.

Hydrotropes which can be used for improving the flow behaviour and the application properties are, for example, ethanol, isopropyl alcohol or polyols. Polyols which are suitable here can have 2 to 15 carbon atoms and at least two hydroxyl groups. Typical examples are:

glycerol alkylene glycols, such as, for example, ethylene glycol, diethylene glycol, propylene glycol, butylene glycol, hexylene glycol, and polyethylene glycols with an average molecular weight of from 100 to 1000 daltons, technical-grade oligoglycerol mixtures with a degree of self-condensation of from 1.5 to 10, such as, for example, technical-grade diglycerol mixtures with a diglycerol content of from 40 to 50% by weight, methylol compounds, such as in particular trimethylolethane, trimethylolpropane, trimethylolbutane, pentaerythritol and dipentaerythritol, lower alkyl glucosides, in particular those with 1 to 4 carbon atoms in the alkyl radical, such as, for example, methyl and butyl glucoside, sugar alcohols having 5 to 12 carbon atoms, such as, for example, sorbitol or mannitol, sugars having 5 to 12 carbon atoms, such as, for example, glucose or sucrose, amino sugars, such as, for example, glucamine.

Solids which can be used are, for example, iron oxide pigments, titanium dioxide or zinc oxide particles and those additionally specified under “UV protectants”. Furthermore, it is also possible to use particles which lead to special sensory effects, such as, for example, nylon-12, boron nitride, polymer particles such as, for example, polyacrylate or polymethyl acrylate particles or silicone elastomers. Fillers which can be used include starch and starch derivatives, such as tapioca starch, distarch phosphate, aluminium starch or sodium starch, octenyl succinate, and pigments which have neither primarily a UV filter effect nor a colouring effect, for example Aerosils® (CAS No. 7631-86-9).

Within the context of the present invention, the solids can advantageously also be used in the form of commercially available oily or aqueous predispersions. The preparations according to the invention usually comprise 0 to 40% by weight of pigments, based on the total weight of the preparation.

Examples of film formers which can be used, for example, for improving the water resistance are: polyurethanes, dimethicones, copolyol, polyacrylates or PVP/VA copolymer (PVP=polyvinylpyrrolidone, VA=vinyl acetate). Fat-soluble film formers which can be used are: e.g. polymers based on polyvinylpyrrolidone (PVP), copolymers of polyvinylpyrrolidone, PVP/hexadecene copolymer or the PVP/eicosene copolymer.

Pearlescence additives which can be used are, for example, glycol distearates or PEG-3 distearate.

Suitable deodorant active ingredients are, for example, odour concealers such as the customary perfume constituents, odour absorbers, for example the sheet silicates described in the patent laid-open specification DE 40 09 347 (AU 7325891), of these in particular montmorillonite, kaolinite, illite, beidelite, nontronite, saponite, hectorite, bentonite, smectite, or also, for example, zinc salts of ricinoleic acid or talc. Antimicrobial agents are likewise suitable for being incorporated. Antimicrobial substances are, for example, 2,4,4′-trichloro-2′-hydroxydiphenyl ether (Irgasan), 1,6-di-(4-chlorophenylbiguanido)hexane (chlorhexidine), 3,4,4′-trichloro-carbonilide, quaternary ammonium compounds, clove oil, mint oil, thyme oil, triethyl citrate, farnesol (3,7,11-trimethyl-2,6,10-dodecatrien-1-ol), ethylhexyl glyceryl ether, polyglyceryl-3 caprylate (TEGO® Cosmo P813, Evonik Goldschmidt GmbH), and the effective agents described in the patent laid-open specifications DE 198 55 934, DE 37 40 186 (U.S. Pat. No. 4,921,694), DE 39 38 140 (U.S. Pat. No. 5,318,778), DE 42 04 321, DE 42 29 707 (AU 4950993), DE 42 29 737, DE 42 38 081, DE 43 09 372 (U.S. Pat. No. 5,718,888), DE 43 24 219 and EP 666 732 (U.S. Pat. No. 5,648,067).

The preparations according to the invention can comprise the deodorant active ingredients in amounts of from 0.1 to 30% by weight, preferably 1 to 20% by weight and in particular 2 to 10% by weight, based on the total weight of the preparation.

Antiperspirant active ingredients are salts of aluminium, zirconium or of zinc. Such suitable antihydrotically effective active ingredients are, for example, aluminium chloride, aluminium chlorohydrate, aluminium dichlorohydrate, aluminium sesquichlorohydrate and complex compounds thereof, e.g. with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, e.g. with amino acids such as glycine. Preference is given to using aluminium chlorohydrate, aluminium zirconium tetrachlorohydrate, aluminium zirconium pentachlorohydrate and complex compounds thereof.

The preparations according to the invention can comprise the antiperspirant active ingredients in amounts of from 1 to 50% by weight, preferably 5 to 30% by weight and in particular 8 to 25% by weight, based on the total weight of the preparations.

### Esterase Inhibitors:

In the presence of perspiration in the axillary area extracellular enzymes—esterases, preferably proteases and/or lipases—are formed by bacteria and these cleave esters present in the perspiration, thereby releasing odour substances. Suitable esterase inhibitors are preferably trialkyl citrates, such as trimethyl citrate, tripropyl citrate, triisopropyl citrate, tributyl citrate and in particular triethyl citrate (Hydagen® CAT, Cognis GmbH, Düsseldorf/FRG). The substances inhibit the enzyme activity and thereby reduce the odour formation. Further substances which are suitable as esterase inhibitors are sterol sulphates or phosphates, such as, for example, lanosterol, cholesterol, campesterol, stigmasterol and sitosterol sulphate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

The preparations according to the invention can comprise the esterase inhibitors in amounts of from 0.01 to 20% by weight, preferably 0.1 to 10% by weight and in particular 0.3 to 5% by weight, based on the total weight of the preparation.

Insect repellents which can be used are, for example, N,N-diethyl-m-toluamide, 1,2-pentanediol or Insect Repellent 3535.

Self-tanning agents which can be used are, for example, dihydroxyacetone and erythrulose.

Suitable preservatives are, for example, phenoxyethanol, formaldehyde solution, parabens, pentanediol or sorbic acid, and the silver complexes known under the name Surfacine®. Further suitable preservatives are the 1,2-alkanediols having 5 to 8 carbon atoms described in WO 07/048,757.

Suitable preservatives are in particular the substances approved according to Annex VI of the EU Directive 76/768/EEC (in the current version), to which reference is hereby explicitly made.

Conditioning agents which can be used are, for example, organic quaternary compounds, such as cetrimonium chloride, dicetyldimonium chloride, behentrimonium chloride, distearyldimonium chloride, behentrimonium methosulphate, distearoylethyldimonium chloride, palmitamidopropyltrimonium chloride, guar hydroxypropyltrimonium chloride, hydroxypropylguar, hydroxypropyltrimonium chloride, or quaternium-80 or else amine derivatives such as, for example, aminopropyldimethicones or stearamidopropyldimethylamines.

Perfumes which can be used are natural or synthetic odorants or mixtures thereof. Natural odorants are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, orange), roots, (mace, angelica, celery, cardamon, costus, iris, thyme), needles and branches (spruce, fir, pine, dwarf-pine), resins and balsams (galbanum, elemi, benzoin, myrrh, olibanum, opoponax). Animal raw materials are also suitable, such as, for example, civet and castoreum. Typical synthetic odorant compounds are products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroycitronellal, lilial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include primarily the terpenes and balsams. It is possible to use mixtures of different odorants which together produce a pleasant scent note. Essential oils of low volatility, which are mostly used as aroma components, are also suitable as perfumes, e.g. sage oil, camomile oil, clove oil, melissa oil, mint oil, cinnamon leaf oil, linden blossom oil, juniper berry oil, vetiver oil, olibanum oil, galbanum oil, labolanum oil and lavandin oil. It is also possible to use bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, fixolide NP, evernyl, iraldein gamma, phenylacetic acid, geranyl acetate, benzyl acetate, rose oxide, romillat, irotyl and floramat alone or in mixtures.

The preparations according to the invention can comprise the perfumes or perfume mixtures in amounts of from 0 to 2% by weight, preferably 0.01 to 1.5% by weight and in particular 0.05 to 1% by weight, based on the total weight of the preparation.

Dyes which can be used are the substances approved and suitable for cosmetic purposes, as are listed, for example, in the publication “Cosmetic Colourants” of the Dyes Commission of the German Research Society, Verlag Chemie, Weinheim, 1984, pp. 81 to 106. These dyes are usually used in concentrations of from 0.001 to 0.1% by weight, based on the total mixture.

Biogenic active ingredients are to be understood as meaning, for example, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, isoflavones, polyglutamic acid, creatine (and creatine derivatives), guanidine (and guanidine derivatives), pseudoceramides, essential oils, peptides, protein hydrolysates, plant extracts, bisabolol, allantoin, panthenol, phytantriol, idebenone, liquorice extract, glycyrrhizidine and idebenone, scleroglucan, β-glucan, santalbic acid and vitamin complexes. Examples of plant extracts are horsechestnut extract, camomile extract, rosemary extract, black and red currant extract, birch extract, rosehip extract, algae extract, green tea extract, aloe extract, ginseng extract, ginkgo extract, grapefruit extract, calendula extract, camphor, thyme extract, mangosteen extract, cystus extract, terminalia arjuna extract, oat extract, oregano extract, raspberry extract, strawberry extract, etc.

The biogenic active ingredients can also include the so-called barrier lipids, examples of which being ceramides, phytosphingosine and derivatives, sphingosine and derivatives, sphinganine and derivatives, pseudoceramides, phospholipids, lysophospholipids, cholesterol and derivatives, cholesteryl ester, free fatty acids, lanolin and derivatives, squalane, squalene and related substances.

Within the context of the invention, the biogenic active ingredients also include anti-acne, such as, for example, benzyl peroxide, phytosphingosine and derivatives, niacinamide hydroxybenzoate, nicotinaldehyde, retinol acid and derivatives, salicylic acid and derivatives, citronellic acid etc., and anti-cellulite, such as, for example, xanthine compounds such as caffeine, theophylline, theobromine and aminophylline, carnitine, carnosine, salicyloyl phytosphingosine, phytosphingosines, santalbic acid etc., as well as antidandruff agents such as, for example, salicylic acid and derivatives, zinc pyrithione, selenium sulphide, sulphur, cyclopiroxolamine, bifonazole, climbazole, octopirox and actirox, etc., as well as astringents, such as, for example, alcohol, aluminium derivatives, gallic acid, pyridoxine salicylate, zinc salts, such as, for example, zinc sulphate, acetate, chloride, lactate, zirconium chlorohydrates, etc. Bleaches such as kojic acid, arbutin, vitamin C and derivatives, hydroquinone, turmeric oil, creatinine, sphingolipids, niacinamide, etc. may likewise be included in the biogenic active ingredients.

Care additives which may be present are, for example, ethoxylated glycerol fatty acid esters, such as, for example, PEG-7 glycerol cocoate, or cationic polymers, such as, for example, polyquaternium-7 or polyglycerol esters.

Superfatting agents which can be used are substances such as, for example, lanolin and lecithin, and also polyethoxylated or acylated lanolin and lecithin derivatives, polyol fatty acid esters, monoglycerides and fatty acid alkanolamides, with the latter simultaneously serving as foam stabilizers.

Solvents which can be used are, for example, aliphatic alcohols such as ethanol, propanol or 1,3-propanediol, cyclic carbonates, such as ethylene carbonate, propylene carbonate, glycerol carbonate, esters of mono- or polycarboxylic acids such as ethyl acetate, ethyl lactate, dimethyl adipate and diethyl adipate, propylene glycol, dipropylene glycol, glycerol, glycerol carbonate or water.

The invention further provides thick-liquid to semisolid and ranging to cream-solid cosmetic and/or dermatological preparations which can be passed onto the consumer in a container suitable for cosmetic sticks or deodorant roll-on container or an atomizer pump container.

The invention further provides cosmetic and/or dermatological preparations which, besides other cosmetic additives, comprise perfume and/or fragrances.

The invention further provides the use of an antiperspirant formulation based on the preparations for application on the human skin, in particular for reducing the formation of perspiration.

Further subjects of the invention arise from the claims, the disclosure content of which, in its entirety, is part of this description.

The cosmetic preparations according to the invention and their preparation and use are described below by way of example, without any intention to limit the invention to these exemplary embodiments. Where ranges, general formulae or compound classes are given below, then these are intended to encompass not only the corresponding ranges or groups of compounds that are explicitly mentioned, but also all part ranges and part groups of compounds which can be obtained by removing individual values (ranges) or compounds. Where documents are cited within the context of the present description, then their content should in its entirety belong to the disclosure content of the present invention.

### EXAMPLES

### 1) Preparation Examples

### 1a) PPG Butyl Ether by Means of DMC Catalysis (According to the Invention)

In a 3 litre autoclave, 400 g of polypropylene glycol monobutyl ether (mass-average molar mass M_{w}=400 g/mol) and 0.03 g of zinc hexacyanocobaltate DMC catalyst are initially introduced under nitrogen, then heated to 130° C. The reactor is evacuated to an internal pressure of 30 mbar in order to remove, by distillation, any volatile ingredients that may be present. To activate the DMC catalyst, a portion of 20 g of propylene oxide is introduced. Following the onset of the reaction and an internal pressure drop, over 80 min, a further 550 g of propylene oxide and 74 g of n-butanol are simultaneously fed in continuously with cooling. Finally, a further 956 g of propylene oxide are metered in over the course of 45 min at 130° C. and a maximum reactor internal pressure of 1.5 bar. The 30-minute afterreaction at 130° C. is followed by the degassing stage. During this, volatile fractions, such as residual propylene oxide, are distilled off in vacuo at 130° C. The finished colourless polyether is cooled to below 90° C. and drawn off from the reactor. It has an OH number of 55 mg KOH/g, an acid number of <0.1 mg KOH/g and, according to GPC, an average molar mass M_{w} of 1100 g/mol or an Mₙ of 1036 g/mol, and a polydispersity M_{W}/Mₙ of 1.06, measured against a polypropylene glycol standard.

### 1b) PPG Butyl Ether by Means of KOH Catalysis (not According to the Invention)

In a 3 litre autoclave, 148 g of n-butanol and 5.6 g of potassium hydroxide are initially introduced under nitrogen and heated to 130° C. 2050 g of propylene oxide are fed in with cooling over ca. 7 h such that, at 130° C., the reactor internal pressure does not exceed 2.5 bar. The 3-hour afterreaction at 130° C. is followed by the degassing stage in order to distil off volatile fractions such as residual propylene oxide in vacuo at 130° C. Then, stripping with nitrogen is carried out for a further hour at 130° C. The still alkaline polyether is cooled to 70° C. and adjusted to a pH of 7 using aqueous sulphuric acid. The product is then stripped with steam and nitrogen at 100-110° C. and max. 50 mbar and the salt residues are separated off by means of a filtration at ca. 60° C.

The finished polyether has an OH number of 53 mg KOH/g, an acid number of <0.1 mg KOH/g and, according to GPC, an average molar mass M_{w} of 1208 g/mol or an Mₙ of 1093 g/mol, and a polydispersity M_{w}/Mₙ of 1.11, measured against polypropylene glycol standard.

### 2) Formulation Examples

### AP/Deodorant Stick Formulations

All data refers to percent by weight (% by weight) unless stated otherwise.

| Example | 2A | 2B |
|---|---|---|
| Stearyl Alcohol | 18 | 18 |
| Hydrogenated Castor Oil | 4 | 4 |
| C₁₂₋₁₅ Alkyl Benzoate | 6 | 6 |
| PPG-14 Butyl Ether (according to preparation | 8 | |
| example 1a) | | |
| PPG-14 Butyl Ether (according to preparation | | 8 |
| example 1b) | | |
| Cyclomethicone | 42 | 42 |
| Talc | 2 | 2 |
| Aluminium Zirconium Tetrachlorohydrex GLY | 20 | 20 |

### 3) Odour Assessment

The AP/Deo stick formulations were investigated by a trained odour panel (4 people). Here, the odour was assessed according to the school grading system from 1 (very good) to 6 (very bad). The first odour assessment of the sticks 2A and 2B was carried out one day after production, the second four weeks after storage in a climatically controlled chamber at 22° C.:

| | 1st assessment | 2nd assessment |
|---|---|---|
| Stick 2A | 2.0 | 2.5 |
| Stick 2B | 2.5 | 3.5 |

Surprisingly, it has also been found that stick 2A exhibited a slightly smaller white rub-off on black cotton fabric than stick 2B. The degree of whitening is therefore surprisingly reduced compared with a standard formulation.

Having thus described in detail various embodiments of the present invention, it is to be understood that the invention defined by the above paragraphs is not to be limited to particular details set forth in the above description as many apparent variations thereof are possible without departing from the spirit or scope of the present invention.

1. A cosmetic and/or dermatological preparation prepared using polyether alcohols, characterized in that, for the preparation of the polyether alcohols,
a) a DMC catalyst is used and
b) the polyether alcohols obtained in this way are not further treated.

2. A cosmetic and/or dermatological preparation according to claim 1, characterized in that a DMC catalyst is used in a concentration of less than 1000 wppm.

3. A cosmetic and/or dermatological preparation according to claim 1, characterized in that the polyether alcohols are pure polypropylene glycol alkyl ethers.

4. A cosmetic and/or dermatological preparation according to claim 1, characterized in that one or more polyether alcohols of the formula (I) are present,
R¹—[O-(A)ₙ-(B)ₘ—H]ₓ (I)
R¹ being a linear or branched alkyl radical having 3 to 22 carbon atoms,
A independently of B being an identical or different ethyleneoxy, propyleneoxy, butyleneoxy, styreneoxy, cyclohexyloxy unit or a unit originating from a glycidyl compound as the result of epoxide ring opening,
B independently of A being an identical or different ethyleneoxy, propyleneoxy, butyleneoxy, styreneoxy, cyclohexyloxy unit or a unit originating from a glycidyl compound as a result of epoxide ring opening,
m being equal to 0 to 20,
n being equal to 1 to 40,
x being an integer from 1 to 6,
and the monomer units A and B can be strung together in their order arbitrarily either blockwise or randomly.

5. A cosmetic and/or dermatological preparation according to claim 1, characterized in that the polyether alcohols are PPG-3 myristyl ether, PPG-11 stearyl ether, PPG-14 butyl ether or PPG-15 stearyl ether.

6. A cosmetic and/or dermatological preparation according to claim 1, characterized in that an additional component is present selected from the group of emollients, emulsifiers and surfactants, thickeners, viscosity regulators, stabilizers, UV photoprotective filters, antioxidants and vitamins, hydrotropes or polyols, solids and fillers, film formers, pearlescence additives, deodorant and antiperspirant active ingredients, esterase inhibitors, insect repellents, self-tanning agents, preservatives, conditioners, perfumes, dyes, biogenic active ingredients, care additives, superfatting agents and/or solvents.

7. A cosmetic and/or dermatological preparation according to claim 1, characterized in that the preparation is thick-liquid to semisolid and ranging to cream-solid and is passed onto the consumer in a container suitable for cosmetic sticks or deodorant roll-on container or an atomizer pump container.

8. A cosmetic and/or dermatological preparation according to claim 1, which further comprises cosmetic additives, perfume and/or fragrances.

9. A method of reducing perspiration on the human skin which comprises of application on the human skin of the cosmetic and/or dermatological preparation of claim 1.

10. The cosmetic and/or dermatological preparation according to claim 2, characterized in that the polyether alcohols are pure polypropylene glycol alkyl ethers.

11. The cosmetic and/or dermatological preparation according to claim 2, characterized in that one or more polyether alcohols of the formula (I) are present,
R¹—[O-(A)ₙ-(B)ₘ—H]ₓ (I)
R¹ being a linear or branched alkyl radical having 3 to 22 carbon atoms,
A independently of B being an identical or different ethyleneoxy, propyleneoxy, butyleneoxy, styreneoxy, cyclohexyloxy unit or a unit originating from a glycidyl compound as the result of epoxide ring opening,
B independently of A being an identical or different ethyleneoxy, propyleneoxy, butyleneoxy, styreneoxy, cyclohexyloxy unit or a unit originating from a glycidyl compound as a result of epoxide ring opening,
m being equal to 0 to 20,
n being equal to 1 to 40,
x being an integer from 1 to 6,
and the monomer units A and B can be strung together in their order arbitrarily either blockwise or randomly.

12. The cosmetic and/or dermatological preparation according to claim 11, characterized in that the polyether alcohols are PPG-3 myristyl ether, PPG-11 stearyl ether, PPG-14 butyl ether or PPG-15 stearyl ether.

13. The cosmetic and/or dermatological preparation according to claim 12, characterized in that an additional component is present selected from the group of emollients, emulsifiers and surfactants, thickeners, viscosity regulators, stabilizers, UV photoprotective filters, antioxidants and vitamins, hydrotropes or polyols, solids and fillers, film formers, pearlescence additives, deodorant and antiperspirant active ingredients, esterase inhibitors, insect repellents, self-tanning agents, preservatives, conditioners, perfumes, dyes, biogenic active ingredients, care additives, superfatting agents and/or solvents.
